(19) United States
(12) Patent Application Publication
(10) Pub. No.: US 2022/0031354 A1
(43) Pub. Date: Feb. 3, 2022

(54) Robotic Double Cannulation Cannula

(71) Applicant: Clark Gerhart, Drums, PA (US)

(72) Inventor: Clark Gerhart, Drums, PA (US)

(21) Appl. No.: 16/945,848

(22) Filed: Aug. 1, 2020

(51) Int. Cl. A61B 17/34; A61B 34/30

(52) U.S. Cl. CPC A61B 17/3423; A61B 34/30; A61B 2017/3419; A61B 2034/302; A61B 2017/3425

(57) ABSTRACT

The Robotic Double Cannulation Cannula is a laparoscopic cannula that will be docked to the Si, X or Xi da Vinci Surgical Systems® (Intuitive, Sunnyvale Calif.) and then placed through an EndoWrist 12 mm & Stapler Cannula® (Intuitive, Sunnyvale Calif.). This provides ease of insertion and extraction of surgical materials, such as suture and mesh, along with specimen extraction, without having to undock the robotic arm. Once inserted, the Robotic Double Cannulation Cannula remote center of movement will match the remote center of movement of the EndoWrist 12 mm & Stapler Cannula® (Intuitive, Sunnyvale Calif.) to prevent injury to the abdominal wall musculature. An electrical contact on the intern tip of the Robotic Double Cannulation Cannula will provide electrical grounding to the outer cannula to prevent electrical injury.

### BACKGROUND

### Field of the Invention

A robotic surgery cannula that is able to be attached to the robotic arm of the da Vinci X and XI Bedside Robotic Cart® (Intuitive, Sunnyvale Calif.) and inserted through (double cannulated in) an EndoWrist 12 mm & Stapler Cannula® (Intuitive, Sunnyvale Calif.); while also sealing around the robotic instruments and providing electrical grounding of the robotic instruments. The new cannula will enable active use of robotic instruments both inside and outside of the patient, allow ease of inserting and withdrawing the robotic instruments for inserting or withdrawing surgical devices, such as suture, mesh and rulers, and withdrawing specimens.

### Description of the Related Art

Laparoscopic surgery has been developed in the last 30 years that allows surgeons to operate on internal organs by placing surgical instruments through the abdominal wall via laparoscopic cannulas, typically made of plastic, that are placed through small punctures rather than using large traditional incisions. The abdomen is inflated with carbon dioxide to provide room to work. The cannulas hold the punctures open for surgical instruments to be inserted, while also sealing them to prevent carbon dioxide gas from escaping.
Robotic laparoscopy also uses cannulas to hold open small punctures, through which the new robotic surgical instruments can be placed into the abdomen. These ae typically made of metal and also seal the openings to prevent carbon dioxide from escaping. The robotic arms that control the instruments attach to these metal cannulas. Once attached to the robotic arm the cannulas become inaccessible to bedside assistants for use in the operation, so additional plastic, non-robotic laparoscopic cannulas are placed for assistants to access the abdominal cavity.
To prevent adding additional cannulas for surgical assistants, surgeons have placed the robotic cannulas through larger plastic laparoscopic cannulas, thus using one puncture for two purposes. To access the abdomen, however, the surgical assistant must remove the robotic cannula from the laparoscopic cannula, which remains in place. When the assistant completes their task the robotic, metal cannula is reinserted through the laparoscopic cannula. The process of inserting a robotic cannula through a plastic assistant cannula is referred to as “double cannulation.”
There are two significant limitations to the current double cannulation technique. First, the metal robotic cannulas require contact with the patient to complete the electrical grounding necessary to prevent arcing of electrical current within the abdominal cavity during the use of electrocautery during surgery, which can potentially cause injury to the patient. Double cannulating a metal cannula through a plastic cannula prevents the metal cannula from contacting the patient, preventing grounding and posing a potential hazard from electrical burns within the abdomen of the patient. Second, the metal robotic cannulas are also designed with a remote center, which is a marking on the trocar that denotes the fulcrum around which the robotic instruments are designed to rotate. When the remote center is positioned within the muscle layer of the patient it provides for the least tension on the muscle, preventing injury to the muscle. Double cannulating with a laparoscopic cannula does not allow for proper positioning of the remote center, raising risk of mechanical injury to the abdominal wall musculature.

### SUMMARY OF INVENTION

The Robotic Double Cannulation Cannula is a robotic cannula that will be attached to the arm of the da Vinci X and Xi robotic Bedside Cart® and double cannulated through another robotic trocar, the EndoWrist 12 mm & Stapler Cannula®. Since it is attached to the robotic arm independent of the EndoWrist 12 mm & Stapler Cannula® it can be inserted and removed without displacing the EndoWrist 12 mm & Stapler Cannula® from the abdominal wall.
The Robotic Double Cannulation Cannula will provide grounding to the EndoWrist 12 mm & Stapler Cannula®, and thus to the patient, to prevent electrical injury to the patient.
The Robotic Double Cannulation Cannula is designed with appropriate specifications, so that when it is inserted into the EndoWrist 12 mm & Stapler Cannula® the remote center will remain unchanged, in the appropriate position within the abdominal wall musculature, preventing mechanical injury to the patient.

### BRIEF DESCRIPTION OF THE DRAWINGS

FIG. 1: Robotic Double Cannulation Cannula

The Robotic Double Cannulation Cannula (RDCC) consists of an External Seal (ES) that will seal around 5 mm or 8 mm robotic instruments preventing escape of gas or liquids from the abdomen; an External Fin (EF) that will allow the Robotic Double Cannulation Cannula (RDCC) to be attached to the Robotic Arm (RA) on the da Vinci X and Xi Robotic Bedside Cart® and a Tubular Cannula (TC) with an Internal Metal Ring (IMR) that will provide electrical grounding between the Robotic Double Cannulation Cannula (RDCC) and the Xi EndoWrist 12 mm & Stapler Cannula® (EW).

FIG. 2: Double Cannulation Process

The Robotic Double Cannulation Cannula will be inserted into the EndoWrist 12 mm & Stapler Cannula® (EW) during use. The Tubular Cannula (TC) with attached Internal Metal Ring (IMR) pass into the lumen of the EndoWrist 12 mm & Stapler Cannula® (EW). The External Seal (ES) seal will seal the external opening of the EndoWrist 12 mm & Stapler Cannula® (EW). The External Fin (EF) is external to the EndoWrist 12 mm & Stapler Cannula® (EW) and will attach to the Robot Arm (RA).

FIG. 3: Withdrawn Position

The Robotic Arm (RA) is in the withdrawn position. The Robotic Double Cannulation Cannula (RDCC) is connected to the Robot Arm (RA) by inserting the External Fin (EF) into the Robotic Arm (RA) cannula mount. Once attached, any Robotic Instrument (RI) can be connected to the Robotic Arm (RA) and inserted into the Robotic Double Cannulation Cannula (RDCC). In the withdrawn position shown, the Robotic Instrument (RI) is outside of the patient but is fully engaged with the Robotic Arm (RA) through the Robotic Double Cannulation Cannula (RDCC), so the Robotic Instrument (RI) is ready for active use, such as to grasp surgical mesh, suture, rulers, or specimens, while outside the patient.

FIG. 4: Inserted Position

From the withdrawn position the Robotic Arm (RA) is then positioned such that the Robotic Instrument (RI) and Robotic Double Cannulation Cannula (RDCC) are inserted through the Xi EndoWrist 12 mm & Stapler Cannula® (EW), already in place in the patient's abdominal wall, allowing the Robotic Instrument (RI) to enter the abdominal cavity. The Robotic Double Cannulation Cannula (RDCC) provides a seal so that no gas or liquids escape the abdomen around the Robotic Instrument (RI) and provides electrical grounding. The Robotic Instrument (RI) is fully engaged with the Robotic Arm (RA) for unrestricted use inside the abdomen during the surgical procedure. The positioning of the Remote Center (RC) on the Xi EndoWrist 12 mm & Stapler Cannula® (EW) remains unchanged so that there is no damage to the abdominal wall musculature.

### DETAILED DESCRIPTION OF THE DRAWINGS

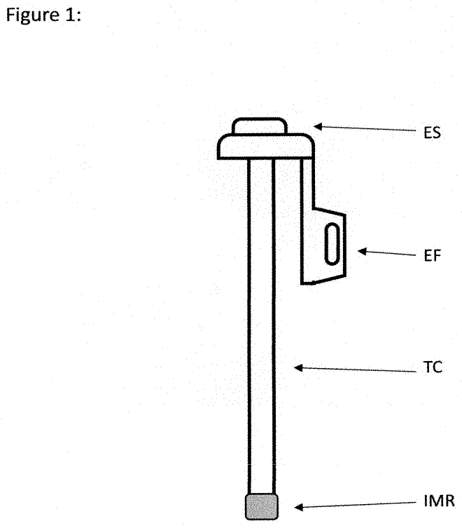

FIG. 1: Robotic Double Cannulation Cannula

The External Seal (ES) is composed of a plastic housing that supports a flexible rubber gasket or valve that will seal around both 5 mm and 8 mm instruments.

The External Fin (EF) is rigid plastic or metal and is attached to the housing of the External Seal (ES) and overhangs the outside of the EndoWrist 12 mm & Stapler Cannula® to a position that duplicates the position of the external fin on the EndoWrist 12 mm & Stapler Cannula®, such that the External Fin (FI) on the Robotic Double Cannulation Cannula (RDCC) is the same distance away from the remote center on the EndoWrist 12 mm & Stapler Cannula®. This preserves the position of the fulcrum of movement of the Robotic Instrument (RI) within the abdominal wall muscle, limiting lateral movement of the cannula at that location and preventing injury to the muscle.

The Tubular Cannula (TC) is a rigid plastic tube attached to the external seal housing. The outside diameter of the Tubular Cannula (TC) is less than 12 mm so that it fits easily into the lumen of the EndoWrist 12 mm & Stapler Cannula®. The inside diameter is 8.5 mm so that it accepts both 8 mm and 5 mm instruments.

The Internal Metal Ring (IMR) is an electrically conductive metal ring that encircles the end of the Tubular Cannula (TC). The outside diameter is the same diameter as the internal diameter of the EndoWrist 12 mm & Stapler Cannula® so that the Internal Metal Ring (IMR) contacts the internal wall of the EndoWrist 12 mm & Stapler Cannula®.

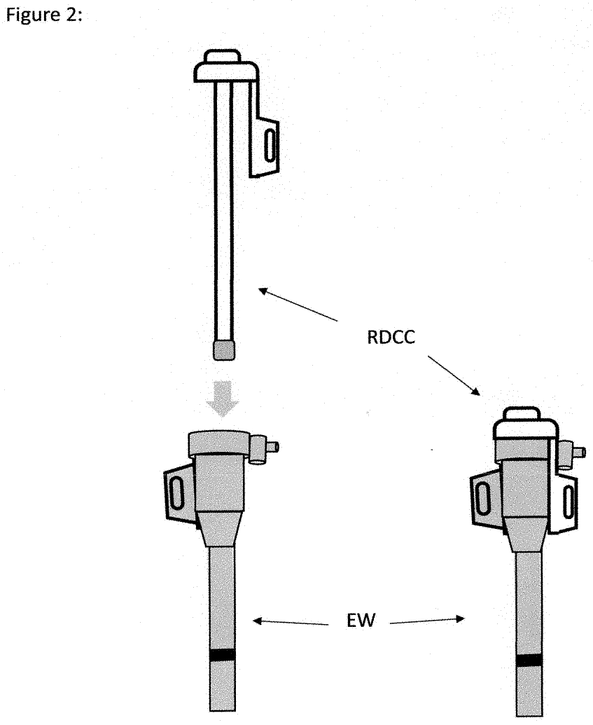

FIG. 2: Double Cannulation Process

The EndoWrist 12 mm & Stapler Cannula® (EW) is currently commercially available from Intuitive, Sunnyvale Calif. It has an external fin allowing it to be connected to the cannula mount on the Robotic Arm (RA). It has a 12 mm cannula seal that prevents gas and liquids from leaking from the abdomen while using 12 mm staplers. It has an external marking indicating the remote center, which is the fulcrum around which the robotic arm rotates. This remote center is placed within the muscle of the abdominal wall to allow for minimal movement and potential damage of the muscle.

The Robotic Double Cannulation Cannula (RDCC) will be inserted into the EndoWrist 12 mm & Stapler Cannula® (EW) during use. The Tubular Cannula (TC) with attached Internal Metal Ring (IMR) pass into the lumen through the seal on the EndoWrist 12 mm & Stapler Cannula® (EW). The External Seal (ES) seals the external opening of the Robotic Double Cannulation Cannula (RDCC) to seal around the robotic instruments preventing gas and liquids from leaking from the abdomen. The External Fin (EF) is external to the EndoWrist 12 mm & Stapler Cannula® (EW) and will attach to the Robot Arm (RA). The Robotic Double Cannulation Cannula (RDCC) can be rotated to avoid interference with the external fin on the EndoWrist 12 mm & Stapler Cannula®. Should it be necessary to use a robotic stapler during an operation, the Robotic Double Cannulation Cannula (RDCC) is simply removed and the external fin of the EndoWrist 12 mm & Stapler Cannula® is attached to the cannula mount on the Robotic Arm (RA) for standard use.

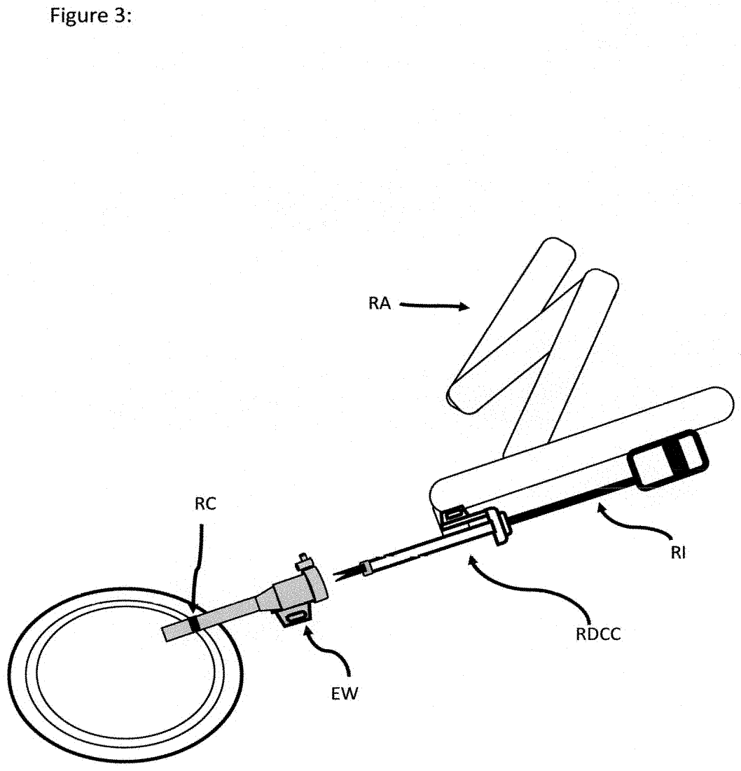

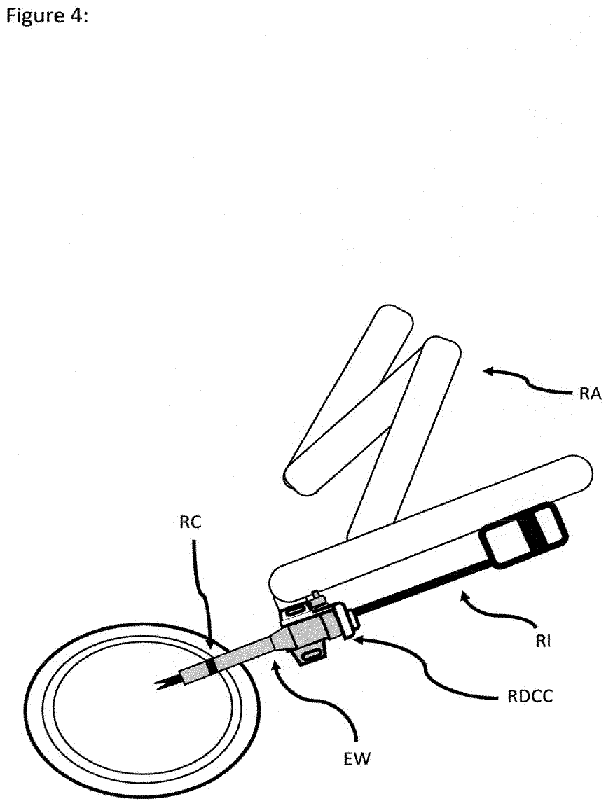

FIG. 3: Withdrawn Position and FIG. 4: Inserted Position

With the Robotic Arm (RA) in the withdrawn position the Robotic Double Cannulation Cannula (RDCC) is connected to the Robot Arm (RA), which makes the arm active for use with any Robotic Instrument (RI). This allows the surgeon to control the instrument for handling surgical materials outside of the patient.

An example of the benefit of this arrangement is passing suture into the abdomen. In the withdrawn position (FIG. 3), the surgeon uses the master controls at the robotic console to open the jaws of the instrument. Suture is then placed into the jaws of the instrument by the bedside assistant. The surgeon then closes the jaws, grasping the suture. The bedside assistant then maneuvers the Robot Arm (RA), inserting the Robotic Double Cannulation Cannula (RDCC), with the robotic instrument grasping the suture, into the EndoWrist 12 mm & Stapler Cannula® (EW) until reaching the Inserted position (FIG. 4). The Robotic Instrument (RI) holding the suture is now in position within the abdomen and fully engaged with the Robotic Arm (RA) for unrestricted use inside the abdomen during the surgical procedure. The Robotic Double Cannulation Cannula (RDCC) provides a seal so that no gas or liquids escape the abdomen around the Robotic Instrument (RI) and electrical grounding. The positioning of the Remote Center (RC) on the Xi EndoWrist 12 mm & Stapler Cannula® (EW) remains unchanged so that there is no damage to the abdominal wall musculature. Once the suturing is completed the surgeon simply grasps the unused portion of suture and the assistant repositions the Robotic arm to the withdrawn position (FIG. 3). The surgeon opens the jaws of the instrument using the controls at the robotic console and the assistant takes the unused portion of suture.

1. An abdominal access device for robotic laparoscopic surgery comprising;
a tubular cannula; and
an external seal; and
an external fin; and
an internal metal rim.

2. An abdominal access device for robotic laparoscopic surgery as in claim 1, wherein said tubular cannula is of appropriate width to fit through the seal and within the lumen of the EndoWrist 12 mm & Stapler Cannula® (Intuitive, Sunnyvale Calif.).

3. An abdominal access device for robotic laparoscopic surgery as in claim 1, wherein said tubular cannula is of appropriate length so that the remote center of movement will coincide with the remote center of movement on the EndoWrist 12 mm & Stapler Cannula® (Intuitive, Sunnyvale Calif.).

4. An abdominal access device for robotic laparoscopic surgery as in claim 1, wherein said external seal covers the external opening of said tubular cannula and allows passage of robotic laparoscopic instruments into the abdomen, and prevents leakage of gas or fluids from the abdomen.

5. An abdominal access device for robotic laparoscopic surgery as in claim 1, wherein said fin is attached to said external seal, and said fin has appropriate specifications to allow it to be connected to the X and Xi da Vinci Surgical Systems® (Intuitive, Sunnyvale Calif.) cannula mount.

6. An abdominal access device for robotic laparoscopic surgery as in claim 1, wherein said metal rim on the internal end of said tubular cannula is in contact with the internal surface of said EndoWrist 12 mm & Stapler Cannula® (Intuitive, Sunnyvale Calif.).
